(19) United States
(12) Patent Application Publication
(10) Pub. No.: US 2005/0251009 A1
(43) Pub. Date: Nov. 10, 2005

(54) System and method for storing and retrieving a communication session

(75) Inventors: Mark M. Morita, Arlington Heights, IL (US); Steven L. Fors, Chicago, IL (US); Khal A. Rai, Round Lake, IL (US)

Correspondence Address: GE MEDICAL SYSTEM;C/O FOLEY & LARDNER, 777 EAST WISCONSIN AVENUE, MILWAUKEE, WI, 53202-5367, US

(73) Assignee: GE Medical Systems Information Technologies, Inc.

(21) Appl. No.: 10/832,926

(22) Filed: Apr. 27, 2004

(51) Int. Cl. A61B 5/05

(52) U.S. Cl. 600/407

(57) ABSTRACT

A graphical user interface is configured to facilitate storage of an electronic communication session generated between a first networked workstation and a second networked workstation and regarding a medical image. The graphical user interface includes a storage function configured to store the communication session in a computer-readable format which is also used to store the medical image, and to associate the communication session with the medical image so that the communication session is capable of being accessed from the medical image.

### BACKGROUND

The present description relates generally to methods and systems for storing, retrieving, and viewing medical images. In particular, the present description relates to storing and retrieving electronic communication sessions regarding medical images.

In radiology and cardiology departments, multiple radiologists, cardiologists, and/or physicians are often involved in collaboration sessions regarding, for example, the analysis and diagnosis of a particular medical image. In order to facilitate these collaboration sessions and minimize interruptions in workflow, communication tools are available such that screen image information from a radiology image viewing workstation regarding a particular medical image may be shared between two or more radiologists at different workstations. For example, the communication tools allow two or more radiologists to view the same medical image on two or more different workstations using the same image settings and cursor.

The communication tools also allow the radiologists at the different workstations to electronically communicate with each other regarding the medical image using text, voice, and or video messages. However, subsequent review stages may be required regarding the medical images. The electronic communication sessions regarding the medical image may be relevant to these subsequent reviews such that they need to be stored and associated with each medical image for later access. Accordingly, there is need for an improved system and method for storing and retrieving an electronic communication session regarding a medical image.

### SUMMARY

According to a first exemplary embodiment, a graphical user interface is configured to facilitate storage of an electronic communication system generated between a first networked workstation and a second networked workstation and regarding a medical image. The graphical user interface includes a storage function configured to store the communication session in a computer-readable format which is also used to store the medical image, and to associate the communication session with the medical image so that the communication session is capable of being accessed from the medical image.

According to a second exemplary embodiment, a system for storing an electronic communication session regarding a medical image includes memory, including the medical image, a display, and a processor coupled to the memory and the display and operable to execute a storage function. The storage function stores the communication session in the memory in a computer-readable format which is also used to store the medical image, and associates the communication session with the medical image so that the communication session is capable of being accessed from the medical image.

According to a third exemplary embodiment, a device for storing an electronic communication session regarding a medical image includes a first networked workstation. The first networked workstation includes a graphical user interface configured to facilitate the communication session regarding the medical image between a first networked workstation and a second networked workstation. The graphical user interface includes a storage function configured to store the communication session in a computer-readable format which is also used to store the medical image, and to associate the communication session with the medical image so that the communication session is capable of being accessed from the medical image.

According to a fourth exemplary embodiment, a method for storing an electronic communication session generated between a first networked device and a second networked device and regarding a medical image includes providing a storage function configured to store the communication session regarding the medical image in a computer-readable format which is also used to store the medical image. The storage function is also configured to associate the communication session with the medical image so that the communication session is capable of being accessed from the medical image.

### BRIEF DESCRIPTION OF THE DRAWINGS

FIG. 1 illustrates a system configured to store and retrieve an electronic communication session regarding a medical image according to an exemplary embodiment.

FIG. 2 illustrates a graphical user interface configured to facilitate storage and retrieval of an electronic communication session regarding a medical image according to an exemplary embodiment.

FIG. 3 illustrates a graphical user interface configured to facilitate storage and retrieval of an electronic communication session regarding a medical image according to another exemplary embodiment.

### DETAILED DESCRIPTION

Turning now to the FIGURES which illustrate exemplary embodiments, a system and method for storing and retrieving an electronic communication session regarding a medical image are shown. Although the present description is provided primarily in the context of storing and retrieving an electronic communication sessions regarding radiology images, it should be understood that the systems and methods described and claimed herein may also be used with all types of medical images. Further, the term “medical image” as used herein includes the medical image, any associated overlays or layers associated with the medical image (e.g., overlays or layers containing annotations, pathological regions of interest, text, measurements, etc.), or any medical report or other information that may be associated with the medical image. Also, it should be understood that, for example, where a single medical image or electronic communication session are recited, a plurality of medical images or electronic communication sessions are encompassed as well. It should also be understood that a particular example or embodiment described herein may be combined with one or more other examples or embodiments also described herein to form various additional embodiments. Accordingly, the systems and methods described herein may encompass various embodiments and permutations as may be appropriate.

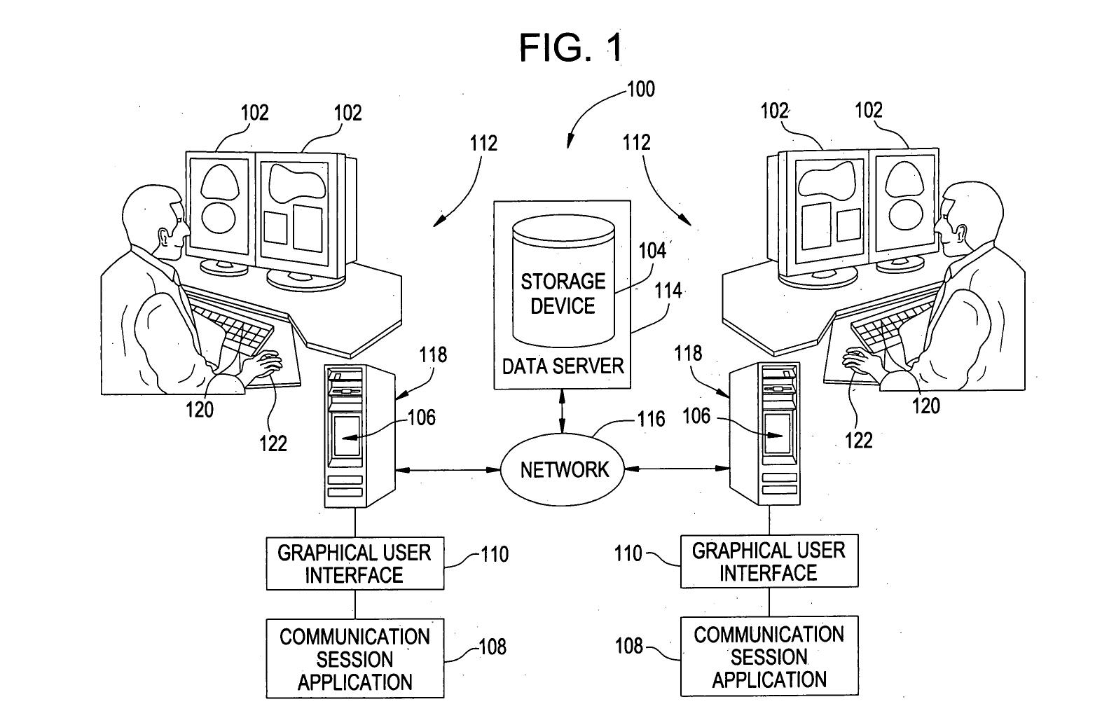

FIG. 1 illustrates a system 100 according to an exemplary embodiment. System 100 includes a display 102, a storage device 104, a processor 106, a communication session application 108, and a graphical user interface (GUI) 110. System 100, broadly described, may be configured to store and retrieve medical images. In particular, system 100 may be configured to store and retrieve an electronic communication session regarding a medical image.

System 100 may be a wide variety of systems for an equally wide variety of uses. For example, system 100 may be any system configured for medical or radiology information management, image communication, and/or image viewing purposes. In one embodiment, system 100 may be a Picture Archiving and Communication System (PACS) used to facilitate the transfer and viewing of medical images and associated data. In another embodiment, system 100 may be a PACS system for viewing radiology images.

System 100 may be configured as a part of a network of computers or as a stand alone system. For example, in the illustrated embodiment, system 100 is a workstation 112, such as a PACS workstation, coupled to a central data server 114 as part of a network 116 which provides access to medical or radiology images from one more workstations 112. As shown in FIG. 1, workstation 112 includes display 110 and cabinet 118 which houses processor 106, communication system application 108, and GUI 110, while storage device 104 is included in data server 114. In another embodiment, display 102, storage device 104, processor 106, communication session application 108, and GUI 110 may all be included in workstation 112 as a stand-alone system.

System 100 may also be configured to include additional components. For example, while FIG. 1 illustrates each workstation 112 as having two displays 102, additional or fewer displays may be used in other embodiments. System 100 may also include a user input device such as keyboard 120 or mouse 122. In one embodiment, system 100 may include one or more voice or audio input and/or output devices or systems, such as a microphone and/or speaker(s) (not shown), to allow for the input and/or output of, for example, voice data. In another embodiment, system 100 may include a video input device, such as a video camera (not shown), to allow for the input and output of video data.

Display 102 is communicatively coupled to processor 106 and may be configured to provide output to a user in the form of information, which may include, for example, alphanumeric output (e.g., text, numbers, etc.) and graphical image output (e.g., radiology images using various imaging technologies. Display 102 may be any number of suitable displays in any number of suitable configurations. For example, display 102 may be a liquid crystal display (LCD), flat screen display, cathode ray tube (CRT) display, SVGA display, VGA display, etc. In one embodiment, display 102 may be a high resolution display for viewing x-ray, ultrasound, magnetic resonance (MR), and computed tomography (CT) images or the like. Display 102 may be further configured to directly receive input from a user (e.g., touch screen, buttons located adjacent to the screen, etc.).

Storage device 104 is communicatively coupled to processor 106 and may be configured to store, for example, medical images. For example, in the illustrated embodiment, storage device 104 is housed in data server 114 and is configured to store medical images to be accessed by one or more workstations 112 connected as part of network 116. In another embodiment, storage device 104 may be incorporated into system 100 where system 100 is a stand-alone system. Storage device 104 may include any computer-readable storage media suitable for storing medical images for later retrieval, such as, for example, a magnetic storage disk, optical storage disk, etc. Storage device 104 may store medical images according to any computer-readable format or protocol. In one embodiment, storage device 104 is configured to store medical or radiology images using a standard Digital Imaging and Communications in Medicine (DICOM) format, which is an electronic storage format which facilitates the transfer of medical images.

Processor 106 is communicatively coupled to display 102, storage device 104, communication session application 108, and graphical user interface (GUI) 110. Processor 106 may be any processor suitable for processing medical images. Processor 106 may be configured to facilitate storage and retrieval of an electronic communication session by executing a storage function included in GIU 110.

Communication session application 108 is communicatively coupled to processor 106 and may be configured to facilitate an electronic communication session regarding a medical image between two or more workstations 112. The electronic communication session may be in the form of an electronic “chat” session between two or more workstations 112 via network 116 using, for example, text data, voice data, video data, or another messaging mode to generate electronic messages regarding the medical image. For example, in one embodiment, communication application 108 may be configured to facilitate an electronic communication session in the form of a conversation or collaboration using electronic text messages between two or more radiologists located at two or more different workstations 112 regarding a medical image or other information commonly displayed on display 102 of each workstation 112. In another embodiment, the radiologists at each respective workstation 112 may communicate with each other regarding the commonly displayed medical image using a series of voice data messages where system 100 includes one or more voice or audio input and/or output devices or systems to allow for the input and/or output of voice data. In another embodiment, the radiologists at each respective workstation 112 may communicate with each other regarding the commonly displayed medical image using video images or data where system 100 includes one or more video input devices. In another embodiment, a combination of text, voice, video and/or other messaging modes may be used to generate the electronic communication session regarding the medical image. Communication session application 108 may also be integrated into system 100 such that two or more radiologists may view the same medical image on two or more different workstations 112 using the same image settings and cursor during an electronic communication session. For example, in one embodiment, system 100 may be a PACS system which integrates communication session application 108 such that two or more radiologists located at two or more different workstations 112 may view a radiology image using the same image settings and cursor during an electronic text communication session regarding the image.

GUI 110 is communicatively coupled to processor 106 and is configured to facilitate storage and retrieval of an electronic communication session regarding a medical image. In one embodiment, GUI 110 may be integrated within communication session application 108. In another embodiment, GUI 110 may be a separate application. GUI 110 may include a storage function that is configured to store an electronic communication session regarding a medical image in a computer readable format which is the same format in which the medical image is stored. For example, in one embodiment, system 100 may be a PACS system and the storage function may be configured to store an electronic communication session regarding a medical image in storage device 104 using a DICOM format, where the medical image is also stored in storage device 104 using a DICOM format.

The storage function is further configured to associate the electronic communication session with the corresponding medical image so that the electronic communication session is capable of being accessed from the medical image. For example, in one embodiment, the storage function is configured to store an electronic communication session regarding a medical image as part of the medical image (e.g., as an associated layer merged with the medical image) using a DICOM format such that the electronic communication session is accessed when the medical image is accessed. In another embodiment, the electronic communication session is stored separate from the medical image using a DICOM format, and is associated with the DICOM medical image such that the electronic communication session may be accessed from the medical image once the medical image is accessed (e.g, from an electronic “link” within the medical image).

GUI 110 may also include a retrieval function that is configured to access the contents of an electronic communication session regarding the medical image so that the electronic communication may be reviewed with the medical image. The particular method of retrieval may vary as to the timing of access of the contents of the electronic communication session regarding the medical image. For example, in one embodiment, the retrieval function may be configured to automatically access the contents of the electronic communication session when the medical image it is associated with is accessed from storage device 104. In another embodiment, the retrieval function may be configured to allow a user of system 100 to access the contents of the electronic communication session from the medical image at any time.

The manner in which the contents of the electronic communication system are accessed and presented may also vary with the particular messaging mode used to generate the electronic communication session. For example, in one embodiment, the electronic communication session may include text data regarding the medical image, and the retrieval function may be configured to transmit the text data to display 102 of system 100 so that it may be displayed with the medical image. In another embodiment, the electronic communication session includes voice data regarding the medical image, and the retrieval function may be configured to transmit the voice data to an audio output. In another embodiment, the electronic communication session may include video data regarding the medical image, and the retrieval function may be configured to transmit the video data to display 102 of system 100 so that it may be displayed with the medical image. In yet another embodiment, the electronic communication session may include a combination of text, voice, and/or video data or data from other messaging modes, and the retrieval function may be configured to access the combination of this data for presentation to a user along with the medical image. In another embodiment, GUI 110 may include functionality that allows a user to selectively access or otherwise manipulate a particular portion of the text, voice, video, or other content of the electronic communication session regarding the medical image.

In this way, an electronic communication session regarding a medical image may be stored and associated with the medical image for use in, for example, subsequent review stages that may be required regarding the medical images. The system and method disclosed herein provides direct access to the electronic communication system from the related medical image, which turn provides for improved radiology workflow. Direct access to the electronic communication session also provides subsequent reviewers further insight into the diagnosis of the medical image up to that point, which leads to improved quality in diagnostic review of the medical image.

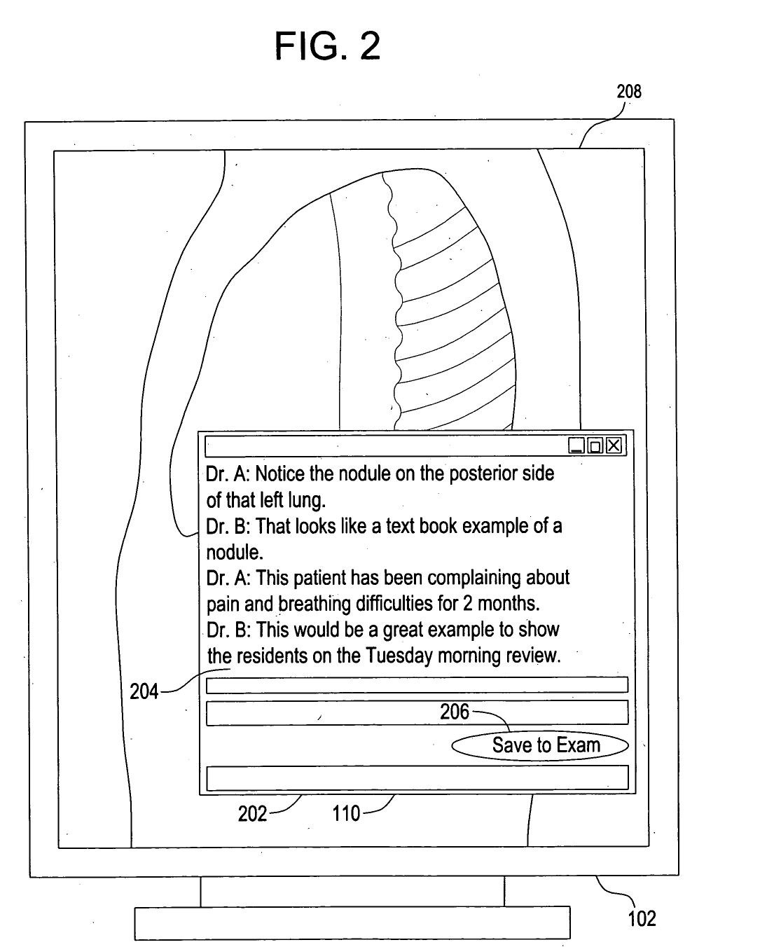

FIG. 2 illustrates a graphical user interface configured to facilitate storage and retrieval of an electronic communication session regarding a medical image according to an exemplary embodiment. In the illustrated embodiment, GUI 110 includes a window 202 displayed on display 102. Window 202 includes a framed area 204 and an icon or “button” 206. Framed area 204 may be configured to display the content of an electronic communication session regarding a medical image 208 displayed on display 102 during the course of the electronic communication session. For example, in one embodiment, framed area 204 may be configured to display electronic text messages entered by two radiologists during an electronic communication session regarding medical image 208. In another embodiment, framed area 204 may be configured to display the video content of an electronic communication session between two radiologists regarding medical image 208. In yet another embodiment, framed area 204 may be configured to display a combination of text and video data or data from other messaging modes during the electronic communication session.

Icon or button 206 may be configured to implement the storage function of GUI 110. Icon or button 206 may be any suitable size, shape, color, etc. For example, in the illustrated embodiment, icon or button 206 is ovular in shape and is labeled “Save To Image.” To implement the storage function at the end of the electronic communication session, a user may, for example, move a cursor over icon or button 206 and “depress” it using a standard “point-and-click” or “point-and double-click” command using mouse 122 (shown in FIG. 1). The storage function may then operate to store the electronic communication in, for example, a DICOM format, in storage device 104 (shown in FIG. 1), and may also associate the electronic communication session with medical image 208.

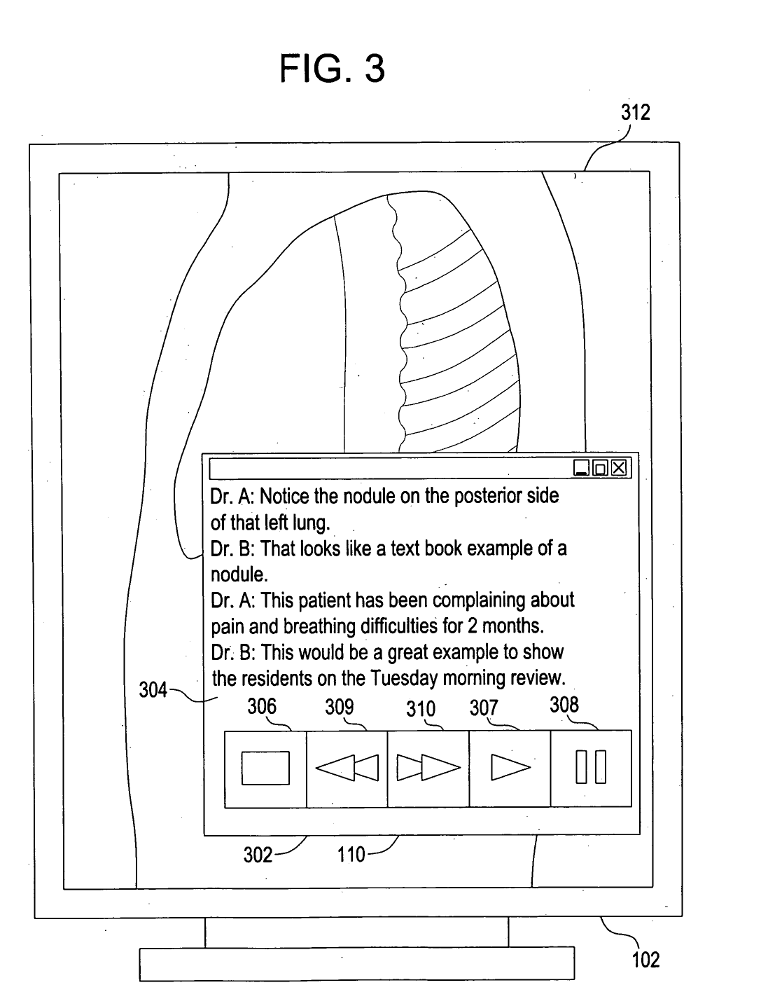

FIG. 3 illustrates a graphical user interface configured to facilitate storage and retrieval of an electronic communication session regarding a medical image according to another exemplary embodiment. In the illustrated embodiment, GUI 110 includes a window 302 displayed on display 102. Window 302 includes a framed area 304 and a series of icons or buttons 306, 307, 308, 309, and 310. Framed area 304 is similar to framed area 204 (shown in FIG. 2) in that may be configured to display the content of an electronic communication session regarding medical image 312. For example, in one embodiment, framed area 304 may be configured to display electronic text messages entered by two radiologists during an electronic communication session regarding medical image 312. In another embodiment, framed area 308 may be configured to display the video content of an electronic communication session between two radiologists regarding medical image 312. In yet another embodiment, framed area 304 may be configured to display a combination of text and video data or data from other messaging modes contained in the electronic communication session regarding medical image 312.

The series of icons or buttons 306-310 may be configured to implement, for example, the storage and retrieval functions of GUI 110. In other embodiments, additional or fewer icons or buttons may be included to implement these functions. Icons or buttons 306-310 may be any suitable size, shape, color, etc. For example, in the illustrated embodiment, icons or buttons 306-310 are essentially square in shape, and each icon or button has an associated symbol or label related to the particular functionality it implements. To implement the functionality of each icon or button 306-310, a user may, for example, move a cursor over one of icons or buttons 306-310 and “depress” it using a standard “point-and-click” or “point-and double-click” command using mouse 122 (shown in FIG. 1).

Icon or button 306 may include a “record” symbol or other similar label and may be configured to implement the storage function of GUI 110. For example, in one embodiment, a user may, for example, move a cursor over icon or button 306 and depress it using mouse 122 (shown in FIG. 1). The storage function may then operate to store the electronic communication in, for example, a DICOM format, in storage device 104 (shown in FIG. 1), and may also associate the electronic communication session with medical image 312.

Icon or button 307 may include a “play” symbol or other similar label and may be configured to implement the retrieval function of GUI 110. For example, in one embodiment, icon or button 307 may be configured to access the contents of an electronic communication session regarding medical image 312 so that electronic text, voice, and/or video data, or data from another messaging mode contained in the electronic communication session may be displayed in framed area 304 and/or sent to an audio output or other output.

Icons or buttons 308, 308, and 310 may be configured to implement additional functionality in GUI 110 that allows a user to selectively access or otherwise manipulate a particular portion of the content of the communication session regarding medical image 312. For example, icon or button 308 may include a “pause” symbol or other label, and may be configured to interrupt access or playback of the electronic text, voice, video, or other content of an electronic communication session. Icon or button 309 may include a reverse, review, or “rewind” symbol or other similar label, and may be configured to allow a user to revisit a previous portion of the text, voice, video, or other content of the electronic communication session. Icon or button 310 may include a “fast forward” or “cue” or other similar label, and may allow a user to advance to a particular portion of the text, voice, video, or other content of the electronic communication session. In other embodiments, other symbols, labels, and/or functions are used with and/or are implemented by icons or buttons 306-310.

The construction and arrangement of the elements described herein are illustrative only. Although only a few embodiments have been described in detail in this disclosure, it should be understood that many modifications are possible without materially departing from the novel teachings and advantages of the subject matter recited in the claims. Accordingly, all such modifications are intended to be included within the scope of the methods and systems described herein. The order and sequence of any process or method steps may be varied or re-sequenced according to alternative embodiments. Other substitutions, modifications, changes, and omissions may be made in the design, operating conditions, and arrangement of the embodiments without departing from the spirit and scope of the methods and systems described herein.

1. A graphical user interface configured to facilitate storage of an electronic communication session generated between a first networked workstation and a second networked workstation and regarding a medical image, the graphical user interface comprising:
a storage function configured to:
store the communication session in a computer-readable format which is also used to store the medical image; and
associate the communication session with the medical image so that the communication session is capable of being accessed from the medical image.

2. The graphical user interface of claim 1, wherein the medical image is a radiology image.

3. The graphical user interface of claim 1, wherein the first workstation and the second workstation are networked using a Picture Archiving and Communication System.

4. The graphical user interface of claim 1, wherein the communication session is stored in a Digital Imaging and Communications in Medicine format.

5. The graphical user interface of claim 1, wherein the communication session includes text data regarding the medical image.

6. The graphical user interface of claim 1, wherein the communication session includes voice data regarding the medical image.

7. The graphical user interface of claim 1, wherein the communication session includes video data regarding the medical image.

8. The graphical user interface of claim 1, wherein the communication session is stored as part of the medical image.

9. The graphical user interface of claim 1, further comprising a retrieval function configured to access the communication session.

10. The graphical user interface of claim 9, wherein the retrieval function is configured to automatically access the communication session when the medical image is accessed.

11. The graphical user interface of claim 9, wherein the communication session includes text data, and wherein the retrieval function is configured to transmit the text data to a display.

12. The graphical user interface of claim 9, wherein the communication session includes voice data, and wherein the retrieval function is configured to transmit the voice data to an audio output.

13. The graphical user interface of claim 9, wherein the communication session includes video data, and wherein the retrieval function is configured to transmit the video data to a display.

14. The graphical user interface of claim 9, further comprising functionality configured to manipulate content of the communication session.

15. A system for storing an electronic communication session regarding a medical image, the system comprising:
memory, including the medical image;
a display; and
a processor coupled to the memory and the display and operable to execute a storage function, wherein the storage function:
stores the communication session in the memory in a computer-readable format which is also used to store the medical image; and
associates the communication session with the medical image so that the communication session is capable of being accessed from the medical image.

16. The system of claim 15, wherein the medical image is a radiology image.

17. The system of claim 15, wherein the system is a Picture Archiving and Communication System.

18. The system of claim 15, wherein the communication session is stored in a Digital Imaging and Communications in Medicine format.

19. The system of claim 15, wherein the communication session includes text data regarding the medical image.

20. The system of claim 15, wherein the communication session includes voice data regarding the medical image.

21. The system of claim 15, wherein the communication session includes video data regarding the medical image.

22. The system of claim 15, wherein the communication session is stored as part of the medical image.

23. The system of claim 15, further comprising a retrieval function configured to access the communication session.

24. The system of claim 23, wherein the retrieval function is configured to automatically access the communication session when the medical image is accessed.

25. The system of claim 23, wherein the communication session includes text data, and wherein the retrieval function is configured to transmit the text data to the display.

26. The system of claim 23, wherein the communication session includes voice data, and wherein the retrieval function is configured to transmit the voice data to an audio output.

27. The system of claim 23, wherein the communication session includes video data, and wherein the retrieval function is configured to transmit the video data to the display.

28. The system of claim 23, further comprising functionality configured to manipulate content of the communication session.

29. A device configured to facilitate storage of an electronic communication session regarding a medical image, the device comprising:
a first networked workstation; the first workstation including a graphical user interface configured to facilitate storage of the communication session regarding the medical image between the first networked workstation and a second networked workstation; the graphical user interface including a storage function configured to:
store the communication session in a computer-readable format which is also used to store the medical image; and
associate the communication session with the medical image so that the communication session is capable of being accessed from the medical image.

30. The device of claim 29, wherein the medical image comprises a radiology image.

31. The device of claim 29, wherein the first workstation and the second workstation are networked using a Picture Archiving and Communication System.

32. The device of claim 29, wherein the communication session is stored in a Digital Imaging and Communications in Medicine format.

33. The device of claim 29, wherein the communication session includes text data regarding the medical image.

34. The device of claim 29, wherein the communication session includes voice data regarding the medical image.

35. The device of claim 29, wherein the communication session includes video data regarding the medical image.

36. The device of claim 29, wherein the communication session is stored as part of the medical image.

37. The device of claim 29, wherein the graphical user interface further includes a retrieval function configured to access the stored communication session.

38. The device of claim 37, wherein the retrieval function is configured to automatically access the communication session when the medical report is accessed.

39. The device of claim 37, wherein the communication session includes text data, and wherein the retrieval function is configured to transmit the text data to the first display system.

40. The device of claim 37, wherein the communication session includes voice data, and wherein the retrieval function is configured to transmit the voice data to an audio output.

41. The device of claim 37, wherein the communication session includes video data, and wherein the display system is configured to transmit the video data to the first display system.

42. The device of claim 37, wherein the graphical user interface further includes functionality configured to manipulate content of the communication session.

43. A method of storing an electronic communication session generated between a first networked device and a second networked device and regarding a medical image, the method comprising:
providing a storage function configured to:
store the communication session regarding the medical image in a computer-readable format which is also used to store the medical image; and
associate the communication session with the medical image so that the communication session is capable of being accessed from the medical image.

44. The method of claim 43, further comprising providing a retrieval function configured to access the communication session.

45. The method of claim 43, further comprising providing functionality configured to manipulate content of the communication session.
